(19) United States
(12) Patent Application Publication
(10) Pub. No.: US 2025/0213144 A1
(43) Pub. Date: Jul. 3, 2025

(54) PATCH-TYPE BODY FLUID SAMPLING AND INSPECTION SYSTEM PROVIDED WITH POROUS MICRONEEDLE, AND METHOD OF MANUFACTURING SAID MICRONEEDLE

(71) Applicants: The University of Tokyo, Tokyo (JP); BNS Medicals Co., LTD., Tokyo (JP)

(72) Inventors: Beomjoon KIM, Tokyo (JP); Leilei BAO, Tokyo (JP); Kai TAKEUCHI, Tokyo (JP); Nobuyuki TAKAMA, Tokyo (JP)

(21) Appl. No.: 18/685,036

(22) PCT Filed: Aug. 19, 2021
(86) PCT No.: PCT/JP2021/030456

§ 371 Date: Feb. 20, 2025

(51) Int. Cl. A61B 5/145; A61B 5/1473

(52) U.S. Cl. CPC A61B 5/14514; A61B 5/1473

(57) ABSTRACT

Provided are an inspection device including a porous microneedle that has high mechanical strength and can allow rapid sampling of an interstitial fluid or the like by capillary force, and a process for manufacturing the microneedle.

An inspection device comprises: a porous microneedle; and a paper substrate sensor having at least one measurement region, in which the microneedle is formed of microspheres of a biodegradable material.

### TECHNICAL FIELD

The present invention relates to a patch-type body fluid sampling and inspection system including a porous microneedle, and a process of manufacturing the microneedle. In addition, the present invention relates to a patch-type body fluid sampling and inspection system having a structure in which a porous microneedle and a paper substrate sensor are integrated.

### BACKGROUND ART

In recent years, a point-of-care test (POCT) device capable of providing health monitoring by rapid diagnosis and inspection in real time has attracted much attention (Non Patent Literature 1). For example, diagnosis using a microchip for health monitoring capable of diagnosing lifestyle diseases called adult diseases such as diabetes, hypertension, cancer, stroke, and heart disease, and infectious diseases such as influenza and COVID-19 from a small amount of blood is expected.

The number of diabetic patients, which is a representative example of lifestyle-related diseases, is estimated to be 10 million in Japan, and 20 million including potential diabetic patients, and is also a major health problem worldwide. Diabetes is a disease that cannot be completely cured by current medical technology, and managing blood glucose levels and preventing the onset of complications are important strategies for patient risk management. Therefore, continuous monitoring of blood glucose levels in daily life is essential for the potential diabetic patients or patients with diabetes.

However, a self-monitoring of blood glucose (SMBG) kit that is currently commercialized performs blood glucose level measurement by puncturing a finger with a needle and collecting blood, and therefore causes pain. In order to reduce the burden on the patient, research on a diagnostic device that tracks blood glucose from a medium such as tears, urine, or sweat has also been advanced, but there are many problems such as inconvenience in wearing, low measurement accuracy, and dirt affecting the measurement result.

On the other hand, an interstitial fluid (ISF) inside the skin has become a promising alternative to blood samples as ISF contains abundant biomarkers (glucose, cholesterol, protein, and the like) that can accurately reflect their concentration in blood (Non Patent Literature 2). Therefore, it is required to develop a convenient and minimally invasive approach for extracting skin ISF for routine self-medical monitoring in routine preventative medicine.

In addition, a minimally invasive biosensor using a needle having a length of about 1 mm or less called a microneedle has attracted attention. A microneedle (MN) array is an effective approach of puncturing up to the dermis layer in order to extract ISF without pain, and a microneedle having a fine hollow structure (hollow needle), a microneedle formed of a hydrogel, a porous microneedle, and the like have been reported so far. However, an MN having a hollow structure formed of metal, silicon, or the like is easily broken, and fragments of the MN remain in the skin, which may damage a human body.

In addition, a biodegradable polymer MN having a porous structure has attracted great attention in recent years. However, there still remain various problems for practical use, such as that the processing is complicated, it takes time to manufacture, and it is difficult to obtain sufficient mechanical strength (strength easy to puncture the skin and the like).

### CITATION LIST

### Non Patent Literatures

Non Patent Literature 1: B. Lei and T. Prow, “A review of microsampling techniques and their social impact”, Biomed. Microdevices, 21, 4 (2019), pp 1-30.
Non Patent Literature 2: B. Martin and H. Derendorf, “Clinical microdialysis: Current applications and potential use in drug development”, Trends Anal. Chem, 25, 7 (2006), pp 674-680.

### SUMMARY OF INVENTION

### Technical Problem

An object of the present invention is to provide an inspection device including a porous microneedle that has high mechanical strength and can allow rapid sampling of an interstitial fluid or the like by capillary force, and a manufacturing method of the microneedle.

In addition, another object of the present invention is to provide an inspection device that includes a porous microneedle and a paper substrate sensor and can measure components in an interstitial fluid in a plurality of measurement regions.

### Solution to Problem

The present inventors have so far conducted studies using a “salt-leaching method” (a method to obtain pores by eluting mixed particles of salt such as NaCl) as a manufacturing method of a porous microneedle formed of a biodegradable polymer. Although the structure of the hole (hole diameter and porosity) can be easily controlled by the salt-leaching method, processing is complicated, and it is difficult to obtain sufficient mechanical strength.

Therefore, as a result of intensive studies, the present inventors have found that a porous microneedle can be manufactured using microspheres of a biodegradable polymer such as polylactic acid by a heat treatment, and the porous microneedle thus obtained has high mechanical strength, thereby completing the present invention.

In addition, the present inventors have found that a porous microneedle array is directly connected to a paper substrate sensor, such that it is possible to provide an inspection device capable of rapidly measuring a blood glucose level or the like in a minimally invasive manner.

That is, the present invention provides the following.

[1] An inspection device comprising:
a porous microneedle; and
a paper substrate sensor having at least one measurement region,
in which the microneedle is formed of microspheres of a biodegradable material.

[2] The inspection device according to [1], in which in the microneedle, the microspheres of the biodegradable material are bonded to each other to form a network of interconnected pores.

[3] The inspection device according to [1] or [2], in which the biodegradable material includes at least one of polylactic acid, polyglycolic acid, a poly(lactide-co-glycolide) copolymer, a PEG copolymer, polyhydroxybutyrate, and ethyl cellulose.

[4] The inspection device according to any one of [1] to [3], further comprising a microneedle substrate, in which the microneedle is bonded to the microneedle substrate.

[5] The inspection device according to any one of [1] to [4], further comprising a channel layer formed between the paper substrate sensor and the microneedle.

[6] The inspection device according to any one of [1] to [3], in which the microneedle and the paper substrate sensor are integrated.

[7] The inspection device according to [1] to [6], in which the microneedle has a break compressive strength of 0.5 N or more as measured under the following conditions.

Conditions: a compressive load is applied to a single microneedle in an axial direction, and a load at a yield point obtained from a load-displacement curve is measured as a break strength.

[8] A process for manufacturing a porous microneedle, said process comprising:
(a) preparing a biodegradable material microsphere solution or suspension containing microspheres of a biodegradable material;
(b) injecting the solution or suspension into a female mold;
(c) drying the solution or suspension to obtain a microneedle precursor; and
(d) heating the microneedle precursor at a predetermined temperature to partially bring the microspheres into a liquid phase or a rubber state and bond the microspheres to each other.

[9] The process according to [8], in which the biodegradable material microsphere solution or suspension is prepared by preparing a solution A in which a biodegradable material is dissolved in an organic solvent, mixing the solution A with an aqueous solution containing a surfactant, and then evaporating and stirring the organic solvent.

[10] A porous microneedle obtained by the process according to [8] or [9].

[11] A microneedle formed of microspheres of a biodegradable material, in which the microspheres of the biodegradable material are bonded to each other to form a network of interconnected pores.

[12] The microneedle according to [11], in which the microneedle has a break compressive strength of 0.5 N or more as measured under the following conditions.

Conditions: a compressive load is applied to a single microneedle in an axial direction, and a load at a yield point obtained from a load-displacement curve is measured as a break strength.

[13] A microneedle array including a plurality of microneedles according to any one of [10] to [12] erected on a microneedle substrate.

### Advantageous Effects of Invention

According to the present invention, it is possible to provide a porous microneedle that has high mechanical strength and can allow rapid sampling of an interstitial fluid or the like by capillary force.

In addition, a process for manufacturing a porous microneedle of the present invention can easily obtain a porous microneedle without requiring complicated steps as in a manufacturing method by a conventional salt-leaching method. Furthermore, in the process for manufacturing a porous microneedle of the present invention, the microspheres are bonded to each other, such that a channel can be formed with a smaller porosity than a porous channel by formation of pores as in the salt-leaching method. In other words, the porous microneedle obtained by the manufacturing method of the present invention has a high-density structure, and can achieve both the mechanical strength and the fluid performance described above.

In addition, according to the present invention, it is possible to provide an inspection device that includes a porous microneedle and a paper substrate sensor and can measure components in an interstitial fluid in a plurality of measurement regions. Such an inspection device can simultaneously detect and measure a plurality of biomarkers such as a blood glucose level and cholesterol. In addition, at the time of the measurement, a reaction in the paper substrate having the sensor structure attached to the skin can be visualized by a color change or the like.

### BRIEF DESCRIPTION OF DRAWINGS

FIG. 1 illustrates a schematic view comparing the form of a microneedle obtained by a salt-leaching method and a microneedle of the present invention.

FIG. 2 illustrates a non-limiting example of an inspection device of the present invention.

FIG. 3 illustrates a schematic view of a method of forming a porous microneedle directly on a paper substrate sensor.

FIG. 4 illustrates an outline of a glucose concentration measuring microneedle patch including the inspection device of the present invention.

FIG. 5 illustrates a preparation process of porous polylactic acid microneedles (PLA MNs) of the present invention.

FIG. 6 illustrates (a) a shape and (b) a dimension of the porous PLA MN after a heat treatment (measurement of dimension is n=5).

FIG. 7 illustrates a result of examining a porous structure of the porous PLA MN obtained in Example 1.

FIG. 8 illustrates a result of measuring a porosity of the porous PLA MN obtained in Example 1.

FIG. 9 illustrates an outline of a test method for measuring an absorption volume of a sample fluid using the porous PLA MNs prepared in Example 1 (left view) and a measurement result (right view).

FIG. 10 illustrates a schematic view of extraction of glucose-loaded ISF from a 1% agarose gel and evaluation of blue color development.

FIG. 11 illustrates results of evaluating extraction and sensing performance of a glucose-loaded sample fluid by the porous PLA MNs prepared in Example 1.

FIG. 12 illustrates an outline of a test method for measuring mechanical strength of the porous PLA MNS prepared in Example 1 (left view) and an obtained load displacement curve (right view).

FIG. 13 illustrates a view obtained by observing a shape of the porous PLA MNs prepared in Example 1 after a test for measuring mechanical strength (left view) and obtained mechanical strength (right view).

FIG. 14 illustrates an outline of an insertion test method for porous PLA MNs using porcine skin (upper view) and results of an insertion test using porous PLA MNs after a heat treatment at different temperatures (middle view and lower view).

FIG. 15 illustrates an outline in which a porous microneedle substrate and a paper substrate sensor are connected by a transparent tape (upper view) and an outline in which the porous microneedle substrate and the paper substrate sensor are connected via a channel layer (lower view).

FIG. 16 illustrates a result of blue color development of the paper substrate sensor according to a concentration of glucose in a phosphate buffer sampled by porous microneedles formed of a 1% agarose gel (left view), and a result of quantitative measurement of a color development density by an image processing program (right view).

FIG. 17 illustrates a comparison of a detection limit concentration of glucose between a patch-type body fluid sampling and inspection system including a porous microneedle and a paper substrate colorimetric sensor or a microneedle electrochemical sensor not using a microneedle.

### DESCRIPTION OF EMBODIMENTS

One embodiment of the present invention is an inspection device comprising a porous microneedle and a paper substrate sensor having at least one measurement region, in which the microneedle is formed of microspheres of a biodegradable material (hereinafter, referred to as “inspection device of the present invention”).

Hereinafter, the inspection device of the present invention will be described in detail with reference to each component.

### 1. Microneedle

### (1) Structure and Properties of Microneedle

A microneedle used in an inspection device of the present invention (hereinafter, also referred to as “microneedle of the present invention”) is porous and formed of microspheres of a biodegradable material. Specifically, the microneedle of the present invention is manufactured using microspheres of a biodegradable material.

In the present specification, the “microspheres” mean spherical fine particles having an average particle size of the order of μm (preferably 1 to 100 μm, more preferably 5 to 30 μm, and still more preferably 10 to 20 μm). Here, the average particle size is usually determined by measurement with an optical microscope.

In the microneedle of the present invention, the microspheres of the biodegradable material are bonded to each other to form a network of interconnected pores. Without intending to be bound by theory, a conventional porous microneedle formed of a biodegradable resin has been manufactured by a salt-leaching method using water-soluble particles such as sodium chloride, but the microneedle of the present invention is different in form (morphology) from the microneedle obtained by such a method. That is, in the salt-leaching method, usually, a biodegradable material and water-soluble particles are mixed, the mixture is filled in a dispenser or the like, droplets are discharged, the droplets are formed into a microneedle shape, thereafter, which is immersed into water and the water-soluble particles are dissolved in water, and when the water-soluble particles are removed, and a site where the water-soluble particles were present becomes a pore, thereby obtaining a porous microneedle (see, for example, WO 2019/176126 A).

On the other hand, in the microneedle of the present invention, a microsphere solution of a biodegradable material is injected into a female mold and dried to obtain a microneedle precursor, and the microneedle precursor is heated to about 150 to 250° C. to bond the microspheres to each other, thereby forming a network of interconnected (communicated) continuous pores. Thus, in the microneedle of the present invention, a rigid pore structure is formed.

In the microneedle of the present invention, it is not necessary that all the microspheres are bonded to each other, but as illustrated in FIG. 7, it is preferable that the microspheres constituting the microneedle precursor are in a state where it can be confirmed by an electron microscope or the like that the microspheres are bonded to each other by heating.

The biodegradable material constituting the microneedle of the present invention includes at least one of polylactic acid, polyglycolic acid, poly(lactide-co-glycolide), a PEG copolymer, polyhydroxybutyrate, and ethyl cellulose.

Here, the microneedle of the present invention may be formed of only the biodegradable material described above, or may contain a raw material (for example, a surfactant such as polyvinyl alcohol, methyl cellulose, sorbitan fatty acid ester, sorbitan monooleate, dodecyl sodium sulfate, or hexadecyltrimethylammonium bromide) used in a method of manufacturing a microneedle of the present invention described below or other additives (for example, carboxymethyl cellulose (CMC) and hyaluronic acid) as trace components within a range not impairing the function of the microneedle of the present invention.

In addition, in the microneedle of the present invention, at least a part of the microneedle may be coated with a coating agent so as not to impair its function. As the coating agent, a material (CMC, hyaluronic acid, or the like) generally used in the present technical field can be used.

The shape of the microneedle of the present invention can have a substantially conical shape, a substantially pyramidal shape, or the like, but a polygonal shape (for example, a substantially pyramidal shape or the like) is more easily penetrated into the skin than the substantially conical shape, which is preferable.

A diameter of a tip portion of the microneedle of the present invention is usually 10 μm to 60 μm. In addition, a diameter or maximum dimension of a base portion is, for example, about 50 μm to 800 μm.

In addition, a height of the microneedle defines a depth of entry into the skin. In the microneedle of the present invention, the height is preferably 300 μm or more and 1,500 μm or less in consideration of reaching the dermis and not stimulating the pain sensation.

An interval when a plurality of microneedles are provided is preferably small in order to absorb the sample of the interstitial fluid, and is preferably an interval of 500 to 5,000 μm.

As for an angle of the tip of the microneedle, the mechanical strength increases when the angle is large, but the force at the time of entry increases when the tip angle is large. When the angle of the tip is 15 to 30°, the force required for the microneedle to enter is less than 0.2 N, which is preferable.

The microneedle of the present invention has strength in which a load at a yield point measured under the following conditions is 0.1 N or more, and preferably 0.5 N or more.

Conditions: a compressive load is applied to a single microneedle in an axial direction, and a load at a yield point obtained from a load-displacement curve is measured as a break strength.

Since the microneedle of the present invention has such high mechanical strength, it is possible to realize the inspection device of the present invention.

As described above, in the microneedle of the present invention, the microspheres are bonded to each other to form a network of interconnected (communicated) continuous pores, thereby forming a rigid pore structure. As a result, the microneedle of the present invention is porous, and can obtain mechanical strength higher than that of a microneedle obtained by a conventional salt-leaching method.

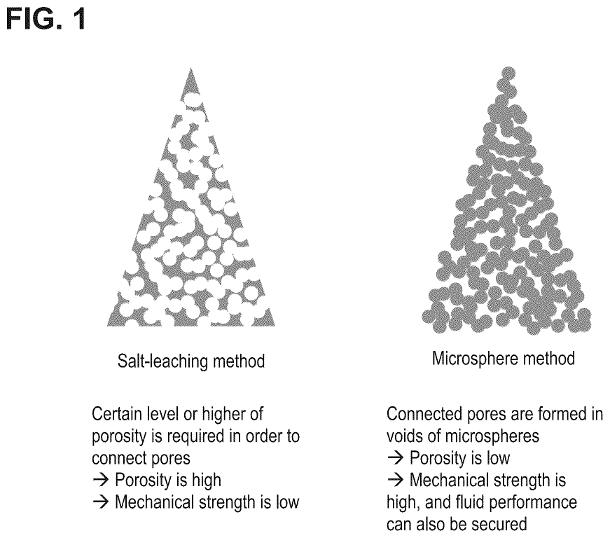

This point will be described in more detail with reference to the schematic view of FIG. 1. That is, in the salt-leaching method, a certain level or higher of porosity is required in order to connect pores, and therefore, a porosity of about 60% is required. Therefore, when a channel is formed, the porosity increases and the mechanical strength decreases. On the other hand, in the microneedle of the present invention obtained by the microsphere method, continuous pores are formed in voids of the microspheres even when the porosity is low. Therefore, a high mechanical strength is obtained while maintaining the channel.

The porosity of the microneedle of the present invention is usually 10 to 40%, and preferably 20 to 30%.

Here, as for the porosity, the mass before and after fluid extraction is compared by a water absorption method Using a porous membrane, and the porosity of the porous microneedle is measured by the following procedure (see P. Liu, et al., J Mater Chem B, 2020).

First, a dry mass (Wary) of the porous membrane is recorded, then the membrane is immersed in deionized (DI) water, and surface water is removed after the absorption is saturated. Thereafter, the mass is measured immediately and recorded as W_{wet}. The porosity is calculated by the following equation.

$$\begin{matrix}[ Math . 1 ] & \\ Porosity ( \% ) = \frac{\frac{{w}_{wet} - {w}_{dry}}{{ρ}_{0}}}{\frac{{w}_{dry}}{{ρ}_{p}} + \frac{{w}_{wet} - {w}_{dry}}{{ρ}_{0}}} , & ( 1 )\end{matrix}$$

In Equation (1), ρₚ is a density of the biodegradable material, and ρ₀ is a density of the DI water (1.0 g/cm³).

The microneedle of the present invention has an excellent water absorption capacity such as a water absorption rate. As also shown in Examples, when the microneedle precursor is heated at a high temperature, the microspheres are partially brought into a liquid phase or a rubber state by a heat treatment, and are bonded to form a robust interconnected micropore network, and the skin interstitial fluid can be efficiently extracted by capillary force.

The microneedle of the present invention has an absorption volume of usually 10 to 150 μL, and preferably 60 to 120 μL as one of indices of water absorption capacity.

Here, the absorption volume is measured by puncturing a microneedle array in which 169 porous PLA MNS are erected on a 1% agarose gel, removing the porous PLA MNs from the gel after 2 minutes, and measuring a weight.

In addition, the microneedle of the present invention usually has an absorption rate of 0.01 to 0.3 μL/min, and preferably 0.2 to 0.3 μL/min per MN.

Here, the absorption rate is measured by puncturing a microneedle array in which 169 porous PLA MNs are erected on a 1% agarose gel, removing the porous PLA MNs from the gel after 2 minutes, and measuring a weight.

The microneedle of the present invention can itself be provided on the paper substrate sensor.

It is also possible to form a microneedle array in which a plurality of microneedles of the present invention are erected on a microneedle substrate so as to bond the microneedles to a paper substrate sensor.

That is, another aspect of the present invention is a microneedle array in which a plurality of microneedles of the present invention are erected on a microneedle substrate (hereinafter, also referred to as “microneedle array of the present invention”).

In the microneedle array of the present invention, the microneedles can be appropriately erected vertically and horizontally. An interval between the microneedles is preferably small in order to absorb the sample of the interstitial fluid, and is preferably an interval of 500 to 5,000 μm.

The microneedle substrate may be formed of the same material as the microneedle or may be formed of a different material.

In one aspect, the microneedle substrate is formed of a film or a hydrocolloidal film containing at least one of a polylactic acid resin, a polyvinyl alcohol resin, a polymethyl methacrylate resin, and a polyurethane resin.

In addition, in another aspect, the microneedle substrate is formed of a biodegradable material. The biodegradable material includes at least one of polylactic acid, polyglycolic acid, a poly(lactide-co-glycolide) copolymer, a PEG copolymer, polyhydroxybutyrate, and ethyl cellulose.

In one preferred aspect, the microneedle substrate is formed of the same biodegradable material as the microneedle, and both are integrally formed.

### (2) Manufacturing Method of Microneedle

Another embodiment of the present invention is a manufacturing method of a porous microneedle (hereinafter, also referred to as “manufacturing method of the present invention”), the manufacturing method including:
(a) preparing a biodegradable material microsphere solution or suspension containing microspheres of a biodegradable material;
(b) injecting the solution or suspension into a female mold;
(c) drying the solution or suspension to obtain a microneedle precursor; and
(d) heating the microneedle precursor at a predetermined temperature to partially bring the microspheres into a liquid phase or a rubber state and bond the microspheres to each other.

The biodegradable material includes at least one of polylactic acid, polyglycolic acid, a poly(lactide-co-glycolide) copolymer, a PEG copolymer, polyhydroxybutyrate, and ethyl cellulose.

A particle size of the microsphere of the biodegradable material is preferably 5 to 30 μm. When the particle size of the microsphere is in this range, it is preferable in terms of achieving both mechanical strength and fluid performance.

A biodegradable material microsphere solution or suspension means a liquid in which microspheres of a biodegradable material are dissolved or dispersed in water or an organic solvent. A biodegradable material microsphere suspension is preferable, and a suspension in which biodegradable material microspheres are dispersed in water is more preferable.

In the manufacturing method of the present invention, preferably, the biodegradable material microsphere solution is prepared by preparing a solution A in which a biodegradable material is dissolved in an organic solvent, mixing the solution A with an aqueous solution containing a surfactant, and then evaporating the organic solvent.

The organic solvent is not particularly limited as long as it is a solvent that dissolves the biodegradable material, and examples thereof include dichloromethane and acetone.

A concentration of the biodegradable material in the solution A is, for example, 0.05 to 0.1% (w/v).

The type of surfactant is preferably polyvinyl alcohol (PVA), carboxymethyl cellulose (CMC), or the like. These surfactants can reduce the surface tension of the solution obtained by mixing the solution A with the aqueous solution, and can stabilize the microspheres to be formed.

In addition, the solution A and/or the aqueous solution containing a surfactant may contain other additives (for example, carboxymethyl cellulose (CMC) and hyaluronic acid) as long as the function of the obtained porous microneedle is not impaired.

The solution obtained by mixing the solution A with the aqueous solution can evaporate the organic solvent by stirring the solution at 500 to 1,500 ppm using a magnetic stirrer or the like at about room temperature.

In the step (b), the biodegradable material microsphere solution or suspension is injected into the female mold. The mold used here is a female micromold prepared from a metal master mold including a large number of microneedles, and polydimethylsiloxane (PDMS), SUS, or the like is preferably used as a material thereof. The shape and size of the metal master-type microneedle can be appropriately determined according to the shape and size of the intended microneedle.

The mold may have only the mold shape of the microneedle to be prepared. In this case, a single microneedle can be obtained. In addition, in a case of manufacturing an inspection device of the present invention in which a microneedle and a paper substrate sensor are integrated as described below, it is preferable to provide such a cavity in a female micromold.

The female micromold may have a desired number of cavities. In addition, for example, cavities can be appropriately provided vertically and horizontally in the female micromold. An interval between the cavities is usually 500 to 5,000 μm and preferably 1,000 to 3,000 μm.

In addition, the cavity can have a shape in which the microneedle substrate and the plurality of microneedles are bonded. In this case, it is possible to obtain a microneedle array in which a plurality of microneedles are bonded to and erected on a microneedle substrate. The micromold itself can have any desired number of cavities. In addition, cavities in the micromold itself can be provided vertically and horizontally as appropriate. The interval between the cavities is preferably 500 to 5,000 μm.

It is preferable that a biodegradable material microsphere solution or suspension is injected into the cavities of the female mold and then left in a vacuum or subjected to centrifugal force to fill the microspheres in the cavities.

In the step (c), moisture, a solvent, and a dispersant are evaporated by drying the biodegradable material microsphere solution or suspension. As a drying method, a temperature can be controlled by providing a pipe in a female micromold, but the entire micromold may be dried in a dryer such as a convection oven.

A drying temperature is preferably 25 to 100° C., and a drying time can be appropriately determined, but is, for example, 1 to 24 hours.

After drying, moisture evaporates from the biodegradable material microsphere solution or suspension to obtain a microneedle precursor composed of biodegradable material microspheres. At this stage, the microneedle precursor may be removed from the mold and subjected to the next step (d). In addition, at this stage, the microneedle precursor may be subjected to the heating step of the next step (d) while being placed in the mold without taking out the microneedle precursor from the mold.

In the step (d), the microneedle precursor is heated at a predetermined temperature. In the microneedle precursor, the individual microspheres maintain the shapes thereof and do not have a form bonded to each other. In the manufacturing method of the present invention, it is important to heat the microneedle precursor at a high temperature to bond the microspheres to each other.

The temperature to be heated here needs to be a temperature at which the microspheres are deformed and bonded to each other, and varies depending on the type of biodegradable resin. For example, in the case of polylactic acid, the temperature is preferably 170 to 200° C. and more preferably 170 to 190° C.

In addition, in the case of polyglycolic acid, the temperature is preferably 170 to 250° C.

In addition, in the case of poly(lactide-co-glycolide) copolymer, the temperature is preferably 50 to 200° C.

In addition, in the case of PEG copolymer, the temperature is preferably 30 to 200° C.

In the case of polyhydroxybutyrate, the temperature is preferably 100 to 200° C.

In the case of ethyl cellulose, the temperature is preferably 80 to 300° C.

The drying time is, for example, 1 to 24 hours.

The porous microneedles obtained by the manufacturing method of the present invention described above are formed of microspheres that are partially in a liquid phase or a rubber state and bonded to each other to form a continuous network of interconnected (communicated) pores, thereby forming a rigid pore structure. The porous microneedle obtained by the manufacturing method of the present invention has higher mechanical strength and excellent water absorption capacity than the microneedle obtained by the conventional method.

### 2. Paper Substrate Sensor

The inspection device of the present invention includes a paper substrate sensor having at least one measurement region.

The capillary action of a liquid on a porous medium can be described by the following Washburn's equation (2), and the present inventors have studied a method for reducing an analysis time for components in the interstitial fluid based on this.

$$\begin{matrix}[ Math . 2 ] & \\ L = \sqrt{\frac{γ R cos Θ t}{2 μ}} & ( 2 )\end{matrix}$$

In Equation (2), L is a flow distance of a liquid, Y is a surface tension of the liquid, R is a radius of a pore, μ is a viscosity of the liquid, θ is a contact angle between the liquid and a porous material, and t is a flow time.

Since there are practical limitations in increasing hydrophilicity and pore size in the porous medium, the inventors have focused on the flow distance (L). Here, since the microneedle is usually prepared on the microneedle substrate, it is conceivable to reduce the thickness of the microneedle substrate to reduce a movement distance. However, the thickness of the microneedle substrate is affected by the manufacturing method, and it is difficult to strictly control the thickness.

Therefore, the present inventors believed that paper can be an appropriate substrate because it is a porous medium having a robust water absorption action, then have conceived a structure in which a microneedle and a paper substrate sensor are integrated. The paper can be several hundred micrometers thick, and further flexibility of the paper increases its usefulness as a patch type for human skin. Once the porous microneedles absorb an analyte, the paper substrate can rapidly transport the analyte to a sensing region.

The paper substrate sensor has at least one measurement region.

As the paper substrate, filter paper is preferably used. The filter paper is preferably filter paper for quantitative analysis defined by JIS P3801, and more preferably filter paper or a nitrocellulose membrane having a thickness of 100 to 500 μm.

The paper substrate sensor has at least one measurement region in a paper substrate such as filter paper, and detects a reaction with a component in an interstitial fluid such as glucose. The measurement is colorimetric measurement mainly using an enzyme, and the concentration and detection of the components in the interstitial fluid can be determined.

The measurement region includes a region where components to be detected (glucose, cholesterol, cortisol, and the like) in the interstitial fluid are measured and a body fluid reaction area where sampling of a body fluid is confirmed.

The region where the components to be detected are measured includes an enzyme that reacts with the components, a peroxide reactant, and a coloring pigment.

For example, in the region where glucose is measured, glucose oxidase (GOx), peroxidase (HRP), and as the coloring pigment, for example, tetramethylbenzidine (TMB) are included.

In the region where cholesterol is measured, cholesterol oxidase is included.

The body fluid reaction area where sampling of a body fluid is confirmed includes cobalt chloride and the like.

One paper substrate sensor may have one measurement region or two or more measurement regions.

In addition, the inspection device of the present invention may comprise a plurality of paper substrate sensors having one measurement region.

### 3. Inspection Device

The inspection device of the present invention includes the microneedle of the present invention and a paper substrate sensor having at least one measurement region as essential components.

The inspection device of the present invention may further comprise a microneedle substrate. In this case, the microneedle is bonded to the microneedle substrate, and the inspection device includes a microneedle array.

Adhesion between the microneedle array and the paper substrate sensor can be performed by adhesion or pressure bonding.

The inspection device of the present invention may further include a channel layer between the paper substrate sensor and the microneedle (or the microneedle array).

The channel layer is formed of a water-absorbing material such as cellulose or filter paper, and has a function of limiting an exudation range of the interstitial fluid according to the measurement region of the paper substrate sensor. In particular, in a case where the paper substrate sensor has a plurality of measurement regions, it is preferable to provide a channel layer.

The channel layer, for example, first attaches a double-side tape with holes (for example, a diameter is about 2 mm) to the microneedle substrate, the holes are filled with cellulose powder. Next, a paper substrate sensor layer can be applied on the opposite side of the double-sided tape to form a fluid channel from the microneedle to the paper substrate sensor.

The inspection device of the present invention may have one or more measurement regions, or two or more measurement regions in one paper substrate sensor.

In addition, the inspection device of the present invention may include a plurality of paper substrate sensors having one measurement region.

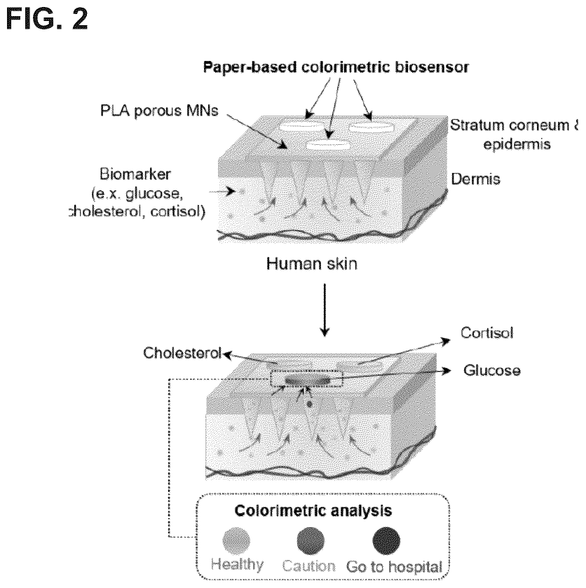

FIG. 2 illustrates a non-limiting example of the inspection device of the present invention. In the inspection device illustrated in FIG. 2, three paper substrate sensors each having a measurement region for glucose, cholesterol, and cortisol are provided.

Such an inspection device having a plurality of measurement regions can simultaneously detect and measure a plurality of biomarkers such as glucose and cholesterol. In addition, at the time of the measurement, a reaction in the paper substrate having the sensor structure attached to the skin is visualized by a color change or the like, optical measurement by a camera or the like and analysis of a color density or the like by software are combined, and information such as a biomarker density or the like can be acquired and analyzed on the spot.

For example, a blood glucose level can be confirmed from a standard color chart, or the blood glucose level can be confirmed in comparison with a standard color change.

In addition, as necessary, the blood glucose level can be quantified by an application such as a portable device, and for example, a person who wants to know the blood glucose level in detail can also take a photograph and quantify the blood glucose level by an application having an image processing function.

Furthermore, it is also possible to monitor in a medical institution or the like in association with another health care monitoring system or the like.

In one aspect of the inspection device of the present invention, a porous microneedle may be provided directly on the paper substrate sensor.

That is, in one aspect of the inspection device of the present invention, the microneedle and the paper substrate sensor are integrated.

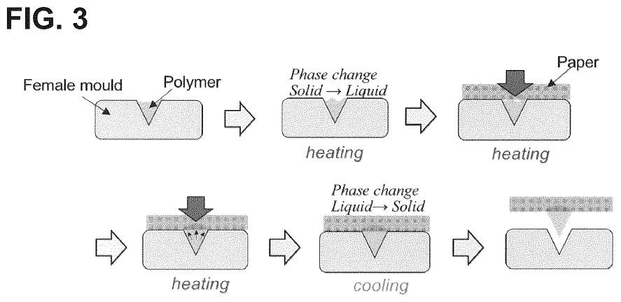

Integrating the porous microneedle with the paper substrate sensor can be achieved by directly molding the porous microneedle onto the paper substrate sensor. A schematic view thereof is illustrated in FIG. 3.

As illustrated in FIG. 3, the biodegradable material solution or suspension is injected into the female mold and solidified to obtain a microneedle precursor, which is then heated in the mold and bonded. Then, while heating, the paper substrate is pressed against the microneedle precursor to integrate them. When the heating step is completed, cooling is performed, and the microneedle is taken out of the mold, such that a structure in which the microneedle and the paper substrate sensor are integrated can be obtained.

The inspection device of the present invention can provide a patch-type body fluid sampling and inspection system that includes a paper substrate sensor of a porous microneedle, is thin, and can easily measure a plurality of components (biomarkers) in an interstitial fluid. In addition, the inspection device of the present invention can allow rapid sampling of an interstitial fluid or the like by capillary force, and can measure components in the interstitial fluid even without external power.

Another aspect of the present invention is a patch-type body fluid sampling and inspection system including the inspection device of the present invention (hereinafter, also referred to as “patch-type body fluid sampling and inspection system of the present invention” or “system of the present invention”).

When a plurality of biomarkers such as glucose and cholesterol are simultaneously detected and measured, the patch-type body fluid sampling and inspection system of the present invention can include a means for visualizing a reaction in the paper substrate having the sensor structure attached to the skin by a color change or the like, combining optical measurement by a camera or the like and analysis of a color density or the like by software, and acquiring and analyzing information such as a biomarker density or the like on the spot.

As such a means, for example, a standard color chart can be provided to confirm the blood glucose level, or a standard color change can be provided to confirm the blood glucose level in comparison with the standard color chart.

In addition, using the patch-type body fluid sampling and inspection system of the present invention, as necessary, the blood glucose level can be quantified by an application such as a portable device, and for example, a person who wants to know the blood glucose level in detail can also take a photograph and quantify the blood glucose level by an application having an image processing function.

Furthermore, it is also possible to monitor in a medical institution or the like in association with another health care monitoring system or the like.

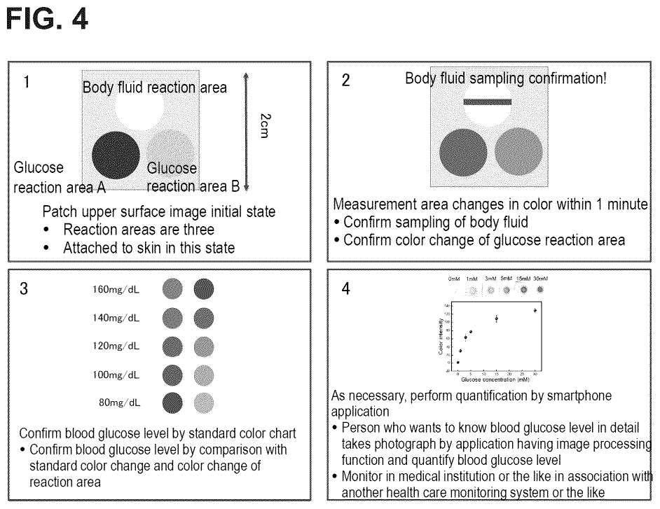

Non-limiting examples of using the inspection device of the present invention and the patch-type body fluid sampling and inspection system of the present invention are illustrated in FIG. 4.

FIG. 4 illustrates an outline of a glucose concentration measuring microneedle patch including the inspection device of the present invention. As illustrated in the upper left view of FIG. 4, the inspection device has three reaction areas of a body fluid reaction area, a glucose reaction area A, and a glucose reaction area B, and is attached to the skin in this state. Here, the type of color developer is different between the area A and the area B.

Thereafter, as illustrated in the upper right view of FIG. 4, the measurement area changes in color within a predetermined time (in this example, within 1 minute). Therefore, sampling of the body fluid is confirmed, and a color change of the glucose reaction area is confirmed.

Next, as illustrated in the lower left view of FIG. 4, the blood glucose level is confirmed by a standard color chart. In addition, the blood glucose level is confirmed by comparison with the standard color chart, a resolution is about 20 mg/dL, and a range of the blood glucose level is about 60 to 300 mg/dL.

Furthermore, as illustrated in the lower right view of FIG. 4, quantification can be performed by an application such as a portable device as necessary. A person who wants to know the blood glucose level in detail can also take a photograph by an application having an image processing function and quantify the blood glucose level (resolution is about 1 mg/dL). In addition, it is also possible to monitor in a medical institution or the like in association with another health care monitoring system or the like.

### EXAMPLES

Hereinafter, the present invention will be described with reference to Examples, but the present invention is not limited thereto.

### 1. Materials

Polylactic acid (PLA): Ingeo Biopolymer 4032D (NatureWorks)

Polyvinyl alcohol (PVA): 363073-500G (Sigma-Aldrich)

Dichloromethane (DCM): 135-02446 (FUJIFILM)

Glucose Sensor Used in Reference Example 1

D-(+)-glucose: 4000535 (Hayashi Pure Chemical Ind., Ltd.)

D-(+)-trehalose dihydrate: T9531-5G (Sigma-Aldrich)

Peroxidase from horseradish: SRE0082-30KU (Sigma-Aldrich)

3,3′,5,5′-Tetramethylbenzidine: 860336-1G (Sigma-Aldrich)

Glucose oxidase: G7141 (Sigma-Aldrich)

Filter paper: Filter paper, Grade 4, Whatman

### 2. Experimental Equipment

Optical microscope: VHX-2000 (Keyence)

Optical microscope (microsphere observation): OMRON VC3000

Force measurement: MX2-500N-FA-V45 (IMADA)

Application of vacuum: vacuum desiccator (AS ONE); Diaphragm type dry vacuum pump DA-30D (ULVAC)

Hot plate: digital hot plate/stirrer PMC-720 (DATAPLATE)

Electronic balance: SECURA 125-1SJP (Sartorius)

### Example 1

### Preparation of Porous Microneedles Using Polylactic Acid Microspheres

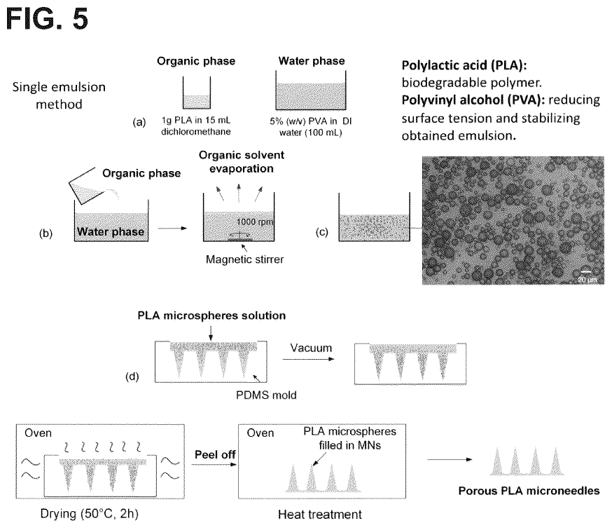

Porous polylactic acid (PLA) microneedles were prepared using PLA microspheres according to the procedure described in the schematic view of the preparation method of the porous microneedle of the present invention illustrated in FIG. 5.

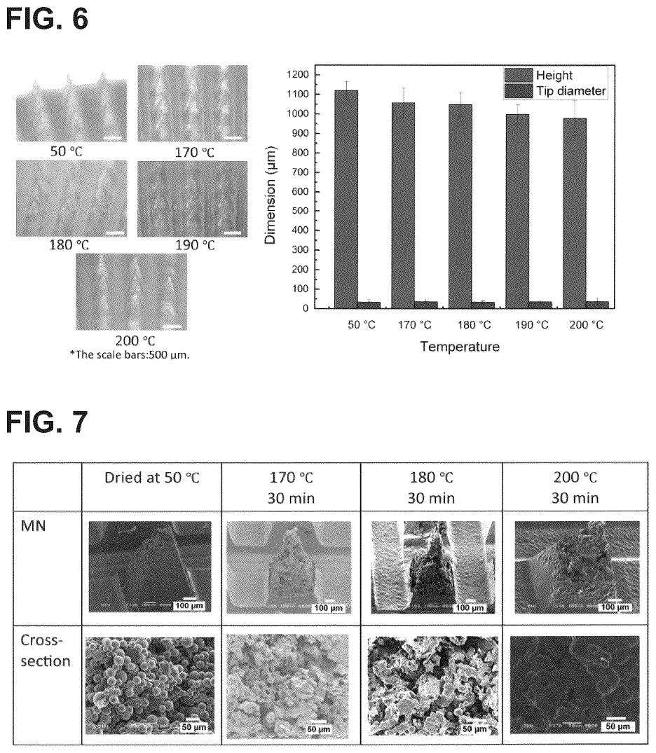

As illustrated in FIGS. 5(a) to 5(c), 6.7% (w/v) of the PLA solution was prepared in dichloromethane (DCM) as an organic phase. Next, the organic phase was blended into an aqueous phase containing 5% (w/v) polyvinyl alcohol (PVA) as a surfactant. The mixture was stirred at 1,000 rpm at room temperature until DCM was evaporated, thereby obtaining PLA microspheres in the PVA solution.

Once the PLA microspheres were formed, the spherical shape was stably maintained in the surrounding environment. The diameter of the microsphere prepared was 15.5±6.9 μm. The diameter of the microsphere was measured by optical microscope observation.

Next, the PLA microsphere solution was injected into a PDMS female mold prepared from a metal master mold consisting of 169 pyramidal microneedles of 1,200 μm in length (FIG. 5(d)). Next, a vacuum was applied to fill the microspheres in the cavities. Next, the entire mold was heated in a convection oven at 50° C. for 2 hours, and the micromold array was peeled off from the mold (FIG. 5(e)). Finally, heat treatments at different temperatures (170, 180, 190, and 200° C.) were applied for 30 minutes to bond the microspheres to each other.

### Example 2

### Evaluation of Porous PLA Microneedle

### (1) Shape and Dimension of Porous PLA Microneedle (MN)

The results of measuring the shape and dimension of the porous PLAMN are illustrated in FIG. 6.

After drying and peeling off from the mold, the height and tip diameter of MN were 1,120.6±47.4 μm and 33.1±14.6 μm, respectively (n=5). As a result of the manufacture, the shape and sharp tip were maintained after the heat treatment, but the height and tip diameter of the MN were slightly reduced. Contraction was attributed to deformation and bonding of PLA microspheres inside MNs (see FIG. 6).

### (2) Porous Structure and Porosity of Porous PLA Microneedle

The porous structure of the MN was examined using a scanning electron microscope (SEM). A single MN and a cross-sectional image are illustrated in FIG. 7.

It can be seen from FIG. 7 that continuous voids were formed after drying at 50° C. for 2 hours, but the PLA microspheres were not bonded to each other. In addition, it can be seen that after the heat treatment at 170° C. for 30 minutes, some of the microspheres started to melt and bond. Since a melting point of PLA used was 170° C., it was considered that the microspheres of PLA started to melt.

In addition, when the heating was performed at 180° C. for 30 minutes, most of the microspheres were completely bonded to each other, ultimately forming a network of micron-sized interconnected pores. In contrast, when the PLA MN was heated to 200° C., the microspheres over-melted and only a small number of interconnected voids were identified.

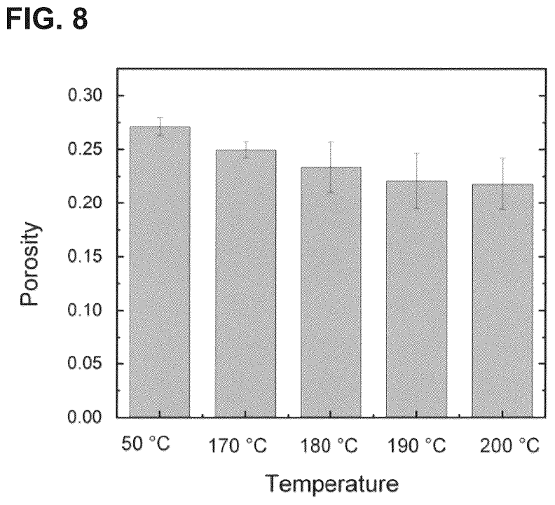

Next, the porosity of the porous PLA MN was measured by comparing the mass before and after fluid extraction by a water absorption method using a porous PLA membrane. First, a dry mass (W_{dry}) of the porous membrane was recorded. Next, the membrane was immersed in deionized (DI) water to remove surface water after the absorption was saturated. Thereafter, the mass was measured immediately and recorded as W_{wet}. The porosity was calculated by the following equation, and the calculation result was graphed (see FIG. 8).

$$\begin{matrix}[ Math . 3 ] & \\ Porosity ( \% ) = \frac{\frac{{w}_{wet} - {w}_{dry}}{{ρ}_{0}}}{\frac{{w}_{dry}}{{ρ}_{p}} + \frac{{w}_{wet} - {w}_{dry}}{{ρ}_{0}}} , & ( 1 )\end{matrix}$$

In the above equation, ρₚ is a density of PLA (1.25 g/cm³), and ρ₀ is a density of DI water (1.0 g/cm³).

The porosity without the heat treatment was 27.2±0.9%. When the heat treatment temperature increased, the porosity gradually decreased as more PLA microspheres melted and were bonded, resulting in reduced continuous porosity inside the microneedles.

### (3) Absorption Capacity of Porous PLA Microneedle

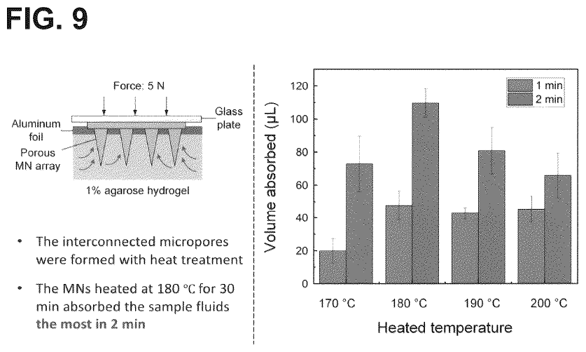

An absorption volume of the sample fluid using the porous PLA MNs prepared in Example 1 was examined using a 1% (w/w) agarose gel coated with an aluminum foil mimicking human skin. A force of 5 N was applied to the MN array and allowed to permeate into the agarose gel (see the left view in FIG. 9).

The MNs heat-treated at 170 to 200° C. passed through the aluminum foil and absorbed the sample fluid. However, the MNs dried at 50° C. penetrated the aluminum foil, but most needles did not maintain the MN structure while remaining in the agarose gel. The reason for this was considered to be weak bonding between the microspheres inside the MNs.

The right view of FIG. 9 illustrates the volume of the sample fluid for 1 and 2 minutes of continuous absorption. The MNs heated to 180° C. absorbed the sample fluid most in 2 minutes.

Next, the extraction of glucose from the glucose-loaded agarose gel was evaluated. An agarose gel loaded with 5 mM glucose at a ratio of 1% (w/w) was coated with an aluminum foil. Glucose detection paper used for the evaluation was prepared by pipetting an enzyme solution containing glucose oxidase (GOx) and peroxidase (HRP) and a chromogenic dye solution using tetramethylbenzidine (TMB), and dried at room temperature.

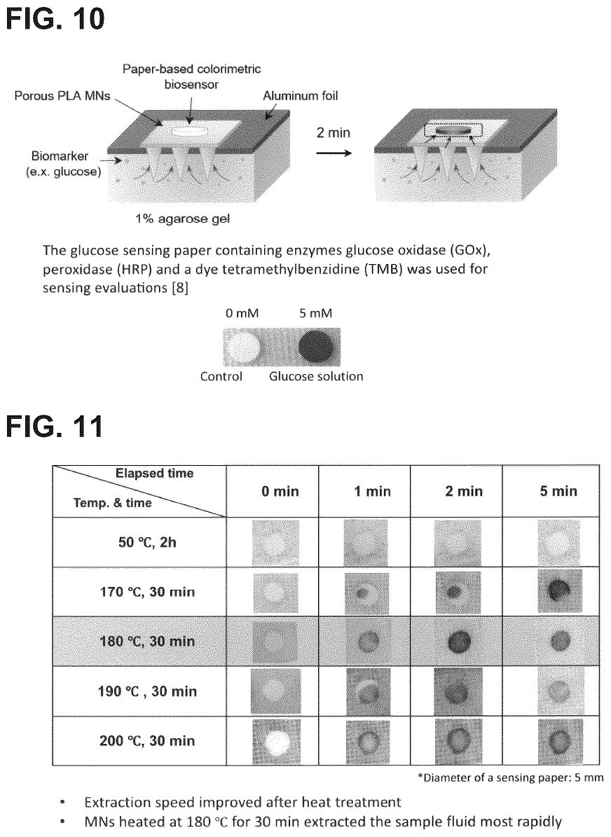

Next, the detection paper was attached to the back surface of the porous MN array (FIG. 10). Next, a pressure was applied with fingers so that the MNs penetrated the model skin. The glucose loaded fluid was extracted and fed to a sensor layer and the reaction was confirmed by the color change of TMB from GOx catalyzed oxidation of glucose (see FIG. 10).

The results of the extraction and glucose sensing performance of the sample ISF of the porous PLA MN are illustrated in FIG. 11. The heat treatment at the melting point or higher of PLA significantly improved the extraction performance of the MN. Furthermore, the PLA MN heat treated at 180° C. for 30 min extracted the sample fluid, which was transported to the sensor layer fastest. As a result, the glucose detector paper turned completely blue within 2 minutes. The reason why the MN hardly absorbed the sample fluid without a heat treatment was that the PLA microspheres were not bonded to each other and did not form stable interconnected pores for feeding the sample fluid. Conversely, it was considered that the heat-treated microspheres melted and were bonded to form robust interconnected micropores, extracting the sample ISF due to the capillary effect.

### (4) Mechanical Strength of Porous PLA Microneedle

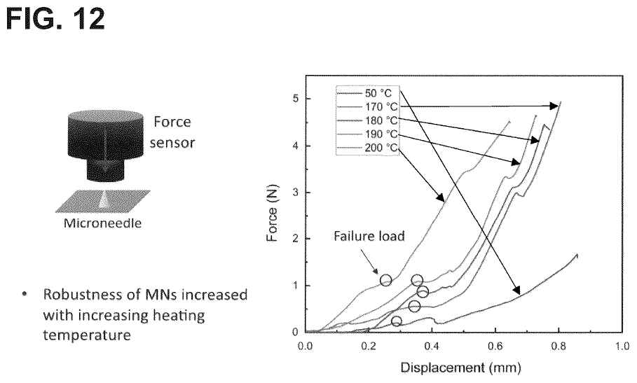

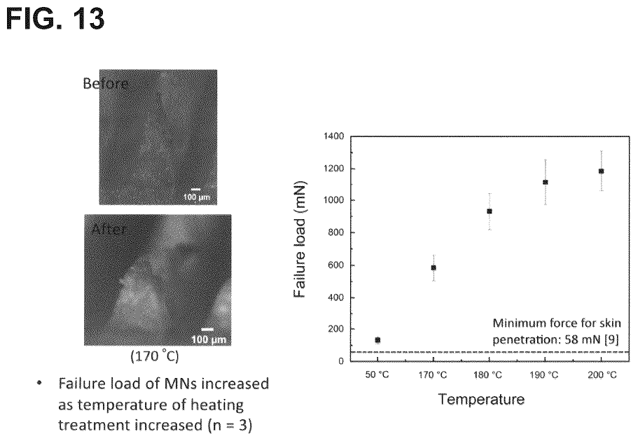

A compressive load was applied to the single MN in an axial direction, and a displacement-load curve was measured. A point at which the load decreased and the displacement increased was defined as a yield point, and the load at the point was defined as a break strength. The results are illustrated in FIGS. 12 and 13. FIG. 12 illustrates an outline of a test method for measuring mechanical strength of the porous PLA MNs prepared in Example 1 (left view) and an obtained load displacement curve (right view).

FIG. 13 illustrates a view obtained by observing a shape of the porous PLA MNs prepared in Example 1 after a test for measuring mechanical strength (left view) and obtained mechanical strength (right view).

### (5) Insertion Test of Porous PLA Microneedles Using Porcine Skin

To verify whether the porous PLA MN has sufficient stiffness to puncture the skin, porcine skin, which is similar to the human skin structure consisting of stratum corneum, epidermis, and dermis, were selected and the insertion test was performed (A. Summerfield, et al., “The immunology of the porcine skin and its value as a model for human skin”, Mol. Immunol, 66, 1 (2015), pp 14-21.).

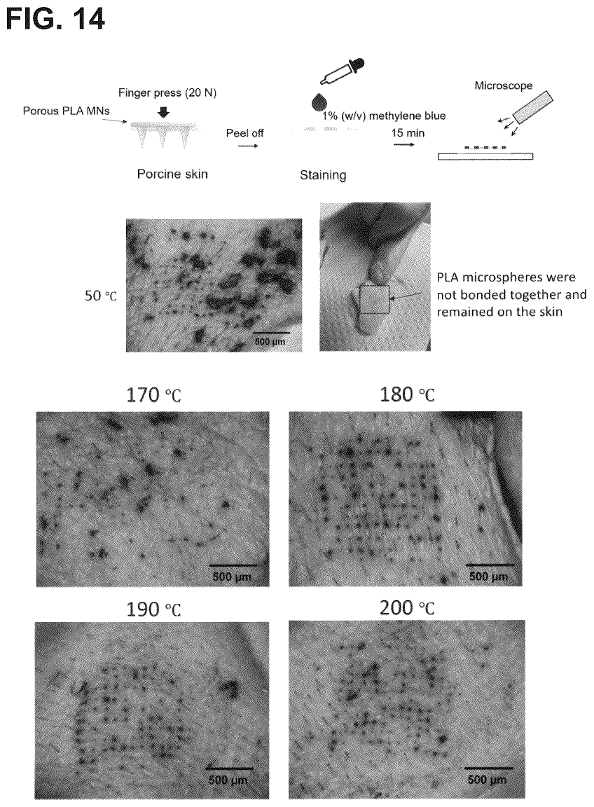

First, porous PLA MNs were inserted into porcine skin by finger pressure and peeled off from the skin. Subsequently, the porcine skin was stained with 1% (w/v) methylene blue for 15 minutes, the methylene blue remaining on the skin was wiped with ethanol, and the transmission site was examined with a light microscope. As illustrated in FIG. 14, the porous PLA MNs, with or without a heat treatment, were able to successfully puncture porcine skin.

However, when the non-heat treated MN patch was detached from the porcine skin after insertion, the overall structure of the MN patch was not maintained and separated (the middle view of FIG. 14). The reason was considered to be that the microspheres in the MN patch lacked adhesion to each other and the patch was separated when detached from the porcine skin. In contrast, most MNs heat-treated at 180 to 200° C. successfully punctured the skin, indicating effective penetration into the skin barrier (see FIG. 14). At the same time, the structure of the MN was intact after peeling from the skin.

### Reference Example 1

Study on Connection between Porous Microneedle Substrate and Paper Substrate Sensor

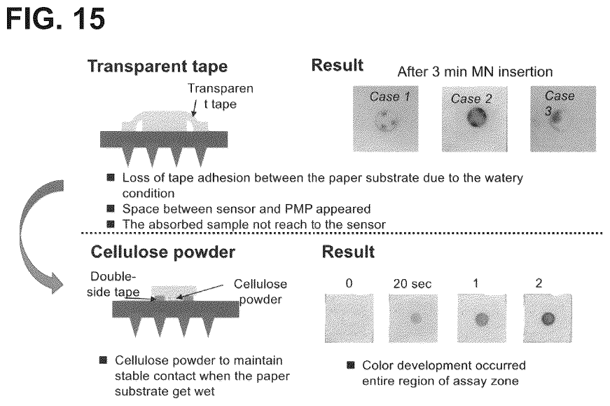

A method for connecting the porous microneedle substrate and the paper substrate sensor using a porous PLGA MN (for details, see Medical Devices and Sensors, Vol.3, Issue 4, e10109, 8, Jul. 2020) manufactured by a salt-leaching method was examined. The method in which the porous microneedle substrate and the paper substrate sensor were connected by a transparent tape (upper view of FIG. 15) and the method in which the porous microneedle substrate and the paper substrate sensor were connected via a channel layer (lower view of FIG. 15) were examined. Here, the channel layer was formed of cellulose powder.

In the connection method by the transparent tape, an adhesive force between the paper substrate and the transparent tape was reduced due to the moisture condition, a space was formed between the porous microneedle substrate and the paper substrate sensor, and the absorbed sample did not reach the sensor (see the right view in the upper view of FIG. 15).

On the other hand, in the method in which the porous microneedle substrate and the paper substrate sensor were connected via the channel layer, it was found that when the paper substrate was wetted, the cellulose powder maintained stable contact, such that color development occurred in the entire range of the reaction area (see the right view of the lower view of FIG. 15).

In the present reference example, the porous PLGA MN manufactured by the salt-leaching method is used as a porous microneedle, but as described in the present specification, the porous microneedle of the present invention has a water absorption capacity equal to or higher than that of a microneedle obtained by the salt-leaching method, and therefore, even when the porous microneedle of the present invention is used together with the glucose sensor, the same result as described above can be obtained.

### Reference Example 2

A concentration of glucose was measured using the glucose sensor and the porous PLGA MN manufactured by a salt-leaching method (for details, see Medical Devices and Sensors, Vol. 3, Issue 4, e10109, 8, Jul. 2020).

In the experiment, a skin model in which an agarose gel embedded in a PBS buffer was covered with an aluminum foil was used. The porosity of the porous PLGA MN was 65% and the insertion strength of the MN was 20 N.

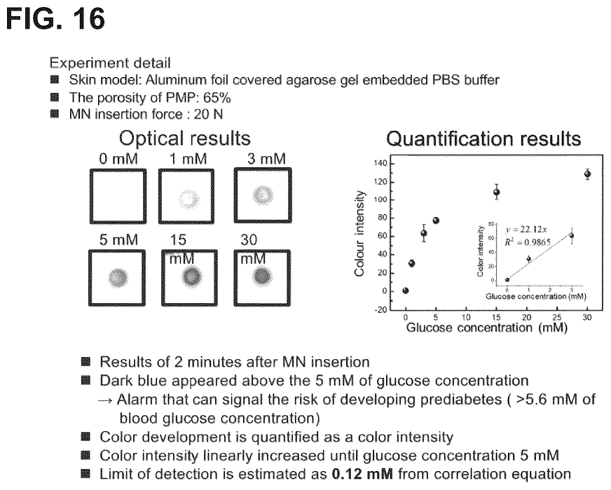

Scanning images and color intensities of assay reactions after 2 minutes of insertion into agarose gels with different concentrations of glucose are illustrated in FIG. 16. FIG. 16 illustrates a result of blue color development of the paper substrate sensor according to a concentration of glucose in a phosphate buffer sampled by porous microneedles formed of a 1% agarose gel (left view), and a result of quantitative measurement of a color development density by an image processing program (right view).

As illustrated in FIG. 16, when the concentration of glucose exceeded 5 mM, dark blue color appeared. This can be used as an alarm that can inform the risk of developing prediabetes (blood sugar level of >5.6 mM).

Color development was quantified as a color density and the color intensity increased linearly until the concentration of glucose was 5 mM. A detection limit is estimated to be 0.12 mM from the correlation equation.

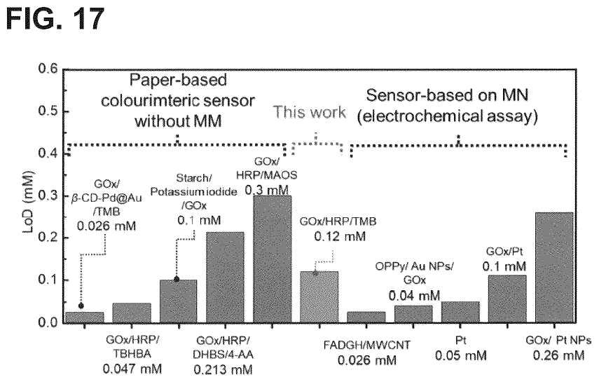

Based on the above results, FIG. 17 illustrates a comparison of a detection limit concentration of glucose between a patch-type body fluid sampling and inspection system including the porous microneedle of Reference Example 2 and a paper substrate colorimetric sensor or a microneedle electrochemical sensor not using a microneedle.

In the present reference example, the porous PLGA MN manufactured by the salt-leaching method is used as a porous microneedle, but as described in the present specification, the porous microneedle of the present invention can obtain higher mechanical strength than the microneedle obtained by the salt-leaching method and has a water absorption capacity equal to or higher than that of the microneedle obtained by the salt-leaching method, and therefore, even when the porous microneedle of the present invention is used together with the glucose sensor, the same result as described above can be obtained.

1. An inspection device comprising:
a porous microneedle; and
a paper substrate sensor having at least one measurement region,
wherein the microneedle is formed of microspheres of a biodegradable material.

2. The inspection device according to claim 1, wherein in the microneedle, the microspheres of the biodegradable material are bonded to each other to form a network of interconnected pores.

3. The inspection device according to claim 1 or 2, wherein the biodegradable material includes at least one of polylactic acid, polyglycolic acid, a poly(lactide-co-glycolide) copolymer, a PEG copolymer, polyhydroxybutyrate, and ethyl cellulose.

4. The inspection device according to any one of claims 1 to 3, further comprising a microneedle substrate, wherein the microneedle is bonded to the microneedle substrate.

5. The inspection device according to any one of claims 1 to 4, further comprising a channel layer formed between the paper substrate sensor and the microneedle.

6. The inspection device according to any one of claims 1 to 3, wherein the microneedle and the paper substrate sensor are integrated.

7. The inspection device according to claims 1 to 6, wherein the microneedle has a break compressive strength of 0.5 N or more as measured under the following conditions,
conditions: a compressive load is applied to a single microneedle in an axial direction, and a load at a yield point obtained from a load-displacement curve is measured as a break strength.

8. A process for manufacturing a porous microneedle, the manufacturing method comprising:
(a) preparing a biodegradable material microsphere solution or suspension containing microspheres of a biodegradable material;
(b) injecting the solution or suspension into a female mold;
(c) drying the solution or suspension to obtain a microneedle precursor; and
(d) heating the microneedle precursor at a predetermined temperature to partially bring the microspheres into a liquid phase or a rubber state and bond the microspheres to each other.

9. The process according to claim 8, wherein the biodegradable material microsphere solution or suspension is prepared by preparing a solution A in which a biodegradable material is dissolved in an organic solvent, mixing the solution A with an aqueous solution containing a surfactant, and then evaporating and stirring the organic solvent.

10. A porous microneedle obtained by the process according to claim 8 or 9.

11. A microneedle formed of microspheres of a biodegradable material, wherein the microspheres of the biodegradable material are bonded to each other to form a network of interconnected pores.

12. The microneedle according to claim 11, wherein the microneedle has a break compressive strength of 0.5 N or more as measured under the following conditions,
conditions: a compressive load is applied to a single microneedle in an axial direction, and a load at a yield point obtained from a load-displacement curve is measured as a break strength.

13. A microneedle array comprising a plurality of microneedles according to any one of claims 10 to 12 erected on a microneedle substrate.
